(19) United States
(12) Patent Application Publication
(10) Pub. No.: US 2008/0255464 A1
(43) Pub. Date: Oct. 16, 2008

(54) SYSTEM AND METHOD FOR DIAGNOSING AND TREATING LONG QT SYNDROME

(76) Inventor: G. Michael Vincent, Salt Lake City, UT (US)

Correspondence Address: J. Rick Tache, Esq.;SNELL & WILMER L.L.P., Suite 1400, 600 Anton Boulevard, Costa Mesa, CA, 92626-7689, US

(21) Appl. No.: 11/733,617

(22) Filed: Apr. 10, 2007

(51) Int. Cl. A61B 5/0452

(52) U.S. Cl. 600/514

(57) ABSTRACT

A system for diagnosing Long QT Syndrome (LQTS) derives a QT/QS2 ratio from an electrical systole (QT) and a mechanical systole (QS2) to detect a prolonged QT interval in a patient's cardiac cycle. A processor acquires the systoles from a microphone and chest electrodes, calculates the QT/QS2 ratio, and outputs the result to a display. The processor may compare the QT/QS2 ratio to a threshold value stored in memory for diagnosing LQTS in the patient. A user interface provides for programming, set-up, and customizing the display. A mode selector allows the system to operate alternatively as a phonocardiograph, a 12 lead electrocardiograph, or a machine for diagnosing LQTS. A related method for diagnosing cardiac disorders such as LQTS includes measuring QT and QS2 during a same cardiac cycle, calculating a QT/QS2 ratio, and comparing the result to a threshold value derived from empirical data. The method may include measuring systoles both at rest and during exercise, and may be used for drug efficacy, dosage optimization, and acquired LQTS causality tests.

### BACKGROUND OF THE INVENTION

1. Field of the Invention

The present invention relates to medical diagnostics for detecting disorders that increase risk for cardiac arrhythmia. More specifically, the invention relates to a diagnostic system and algorithm for detecting long QT syndromes (inherited and acquired) using a combination of electric and acoustic cardiac signals, and particularly, the relationship of the acoustic signal to the QT interval signal.

2. Description of Related Art

Inherited Long QT syndrome (LQTS) is a rare heart disease estimated to affect at least 1 in 5,000 people and result in about 3,000 deaths within the United States each year. The syndrome may lead to tragic, unexpected death, most commonly in children and young adults. In people with LQTS, the heart's electrical system takes too long to recharge, due to abnormal repolarization of the ventricles. This causes an abnormally long delay between electrical excitation (or depolarization) and electrical recovery (or repolarization). The delay can be seen on an electrocardiogram (ECG) as a prolonged QT interval, as shown in FIG. 1. The QT interval is measured from the Q onset point to the end of the T wave, where the Q wave corresponds to the beginning of ventricular depolarization and the T wave end corresponds to the end of ventricular repolarization. The repolarization delay is typically provoked by emotional or physical stress or sudden excitement, and can cause potentially fatal ventricular arrhythmias such as torsade des pointes (TDP), which may degenerate into ventricular fibrillation.

Inherited LQTS results from mutations of genes that encode for proteins that regulate the heart's electrical activity. These proteins make “ion channels” which are microscopic pores through which ions such as potassium, sodium, calcium, etc. pass across cell membranes. A group of inherited diseases that adversely affect these channels have been identified as “ion channelopathies.” LQTS was the first ion channelopathy to be described, recognized by prolongation of the QT interval on the electrocardiogram (ECG), thus, the name of the disorder. Inherited LQTS has been linked to unexpected loss of consciousness, cardiac arrest, or sudden death, primarily in children and young adults.

Drug induced, acquired LQTS is a related disorder. About 50 prescription drugs and several over the counter drugs have the adverse side effect of causing QT prolongation and increasing risk for the same arrhythmia (TDP) that occurs in inherited LQTS. These drugs interact with the encoded protein of one of the known LQTS genes (primarily LQT2, encoding for the IKr channel) impairing the function of the cardiac ion channel and delaying repolarization of the ventricles. Thus acquired LQTS can lead to potentially lethal cardiac arrhythmias through precisely the same mechanism as inherited LQTS. While only a small percentage of the millions of persons in the United States who receive prescriptions for such drugs have a dangerous arrhythmia, the absolute number of patients with this adverse consequence is significant. The FDA, the medical and legal professions, and the pharmaceutical industry are all concerned about this problem.

It is estimated that there are approximately 60,000 inherited LQTS patients in the United States. Extrapolating this value, it may be expected that there are at least 1.5 million LQTS patients worldwide. The actual number is likely even higher than estimated since the ECG criteria upon which the estimates have been based have an important degree of inaccuracy. One source of inaccuracy is that about 60% of those inheriting the genetic defect never show symptoms and often do not receive an ECG. Consequently, they are unlikely to be identified as LQTS patients. Despite being undiagnosed as having LQTS, these patients will pass the genetic defect on to a percentage of their children who may have symptoms or exhibit a very long QT interval and thus receive a correct diagnosis. A second source of inaccuracy is that most diagnoses are made by ECG criteria, using the QTc interval (QT interval corrected for heart rate). Furthermore, there are interpretational limitations inherent in an ECG, including the fact that about 30% of LQTS patients have QT intervals in the same range as normal patients and thus are regularly overlooked. Even though molecular genetic testing is available for diagnosis, the current cost of the test and an unacceptable degree of false negatives generated by test have prevented the test from being used as a means for screening populations. As a result, in any given patient it is often difficult to determine who is affected with LQTS and who is not, due to this overlap and imprecision of the QT intervals.

The ECG is the most commonly used test for the diagnosis of LQTS, identified by a prolongation of the QTc interval. The QTc interval is a calculated interval that represents the QT interval corrected for heart rate. One problem with ECG diagnosis is that the QTc interval is neither completely sensitive nor specific for the diagnosis of LQTS. In other words, the length of the QT (or QTc) interval measured on an ECG, in and of itself, does not always provide a definite indication of LQTS. Those intervals vary with each patient and there is a range of normal values and a wide range of overlap of QTc values between patients having LQTS and patients who do not have LQTS. Thus, a reliable diagnosis is often difficult and the disease may remain unrecognized even after an ECG has been obtained and interpreted. Since LQTS is a potentially fatal disease, those who are misdiagnosed as not having LQTS remain at risk for sudden death because they are not treated.

Thus, the present data on LQTS is likely diluted with over and under diagnoses because about 30% of inherited LQTS patients (estimated at 450,000 patients worldwide) and 60% of the roughly 5.6 billion persons without LQTS (about 3.3 billion people worldwide) have QT intervals in the same (overlap) range and thus are hard to distinguish solely based upon QTc criteria. Added to this are the very large number of patients who receive QT prolonging drugs.

The wide range of QTc intervals in patients in general, and the imprecision of the QTc interval to detect which patients might be at risk for the QT prolonging side effect and for increased risk of TDP makes identification of at-risk patients difficult. These numbers highlight a significant medical problem and the tremendous advantage that a better diagnostic tool could provide.

### SUMMARY OF THE INVENTION

The present invention discloses a system and process for improved identification of abnormal cardiac electrophysiological properties that predispose persons to dangerous cardiac arrhythmias that can lead to unexpected loss of consciousness (syncope), sudden death, and cardiac arrest in LQTS patients. Inherited LQTS is the prototype inherited cause, most common in children and young adults, while acquired LQTS is most commonly caused by prescription medications and can affect patients of all ages. An important feature of the invention is the addition of the QS2 interval as a means for evaluating the QT interval length in each patient, the combination of these intervals providing a significantly improved method for diagnosing LQTS.

The system combines electrocardiograph signals and phonocardiograph signals to detect a prolonged QT interval in a patient's cardiac cycle by comparing the QT interval to the QS2 interval, using a QT/QS2 ratio. The system includes a processing module coupled to electrodes and to at least one microphone. The electrodes transmit the ECG signal to the processor, allowing detection of electrical systole (QT), and may be connected to a patient in a manner similar to connections used for an ECG machine. The microphone detects the second heart sound, caused by closure of the aortic and pulmonic valves and allows determination of the mechanical systole (QS2), and may be located for optimal acoustic measurement of the second heart sound. The processing module receives input signals from the electrodes and microphone, and calculates a ratio of time intervals (QT/QS2) that reflect the electrical and mechanical properties of the heart. The QT/QS2 ratio reflects the normalcy or abnormalcy of the duration of cardiac electrical repolarization properties and has value for the diagnosis and management of, and the risk prediction for arrhythmias of a number of conditions that predispose patients to sudden death.

The system may also record the QT/QS2 ratio in memory, and display the ratio as a diagnostic result on a display device. A user interface may be provided for programming, set-up, and customizing the display. In another embodiment, the system may include a mode selector to allow a user to operate the system as a 12 lead ECG machine, or as a device for calculating QT/QS2.

A related process according to the invention facilitates diagnosis of an abnormal condition such as LQTS by a cardiologist or other health care provider. The process detects for LQTS in a patient by measuring, during the same cardiac cycle, an electrical systole QT and a mechanical systole QS2, then calculating a QT/QS2 ratio, and displaying the result of the calculation. The process may include comparing the ratio to empirical data, and displaying a diagnostic result if the calculated ratio exceeds a predetermined LQTS threshold. Testing during diagnosis may be done both at rest and during a structured exercise protocol. When done during exercise, the process may include calculating a corrected QT value, the QTc, for additional diagnostic value.

In other related methods, the invention may be used to guide treatment of LQTS patients by obtaining QT/QS2 ratios after administration of a drug, to evaluate the effectiveness of the drug, or to optimize dosage. The invention may also provide a method for testing efficacy of drugs proposed for treating LQTS, and a method for evaluating whether a drug poses a risk of causing acquired LQTS.

### BRIEF DESCRIPTION OF THE DRAWINGS

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims. The invention will be better understood upon consideration of the specification and the accompanying drawings, in which like reference numerals designate like parts throughout the figures, and wherein:

FIG. 1 is a graphical representation of one cardiac cycle showing the QT interval, as typically recorded by an ECG.

FIG. 2 is a graphical representation of one period of a cardiac cycle showing the S1 and S2 sound waves, as typically recorded by a phonocardiograph.

FIG. 3 is a block diagram of one embodiment of a system according to the invention for diagnosing LQTS.

FIG. 4 is a frontal view of a human heart showing the position of the pulmonary artery.

FIG. 5 is a frontal view of a human torso showing an approximate location for placing a microphone, and approximate locations for placing the V1-V6 electrodes, for diagnosing LQTS according to the invention.

FIG. 6 is a block diagram of a second embodiment of a system according to the invention for diagnosing LQTS.

FIG. 7 is a block diagram of one embodiment of a QT/QS2 module according to the invention.

FIG. 8 is a display of synchronized electrocardiographs and phonocardiographs illustrating a method according to the invention for diagnosing LQTS. The display shows a normal QT response (A) and an abnormal QT response (B), both at rest and during exercise.

FIG. 9 is a process flow diagram illustrating one embodiment of a method according to the invention for diagnosing LQTS.

FIG. 10 is a process flow diagram illustrating a second embodiment of a method according to the invention for diagnosing LQTS.

### DETAILED DESCRIPTION OF THE INVENTION

The 12 lead ECG and the ambulatory ECG are the most common devices for detecting cardiac arrhythmia. The phonocardiograph is not commonly used to detect arrhythmias or risk of arrhythmia, but adds very important information concerning the normalcy or abnormalcy of cardiac repolarization duration. The phonocardiograph uses a microphone to detect acoustic waves caused by opening and closure of the heart valves and acceleration and deceleration of blood, and thus transduces acoustic waves into an electrical analogue. The electrocardiograph uses electrodes that directly measure the varying strength of electrical signals transmitted by the heart during depolarization and repolarization. The electrocardiograph produces an ECG, and by using multiple electrodes, it is capable of recording electrical activity occurring throughout a patient's body. In this respect, the electrocardiograph is more versatile than the phonocardiograph, and along with echocardiography and other imaging devices, has replaced the phonocardiograph as a diagnostic tool in recent years. Because the QS2 interval obtained by the phonocardiogram may add importantly to analysis or risk for cardiac arrhythmias, the present invention combines features from both the electrocardiograph and the phonocardiograph into a novel instrument for detecting risk for cardiac arrhythmia, specifically, LQTS.

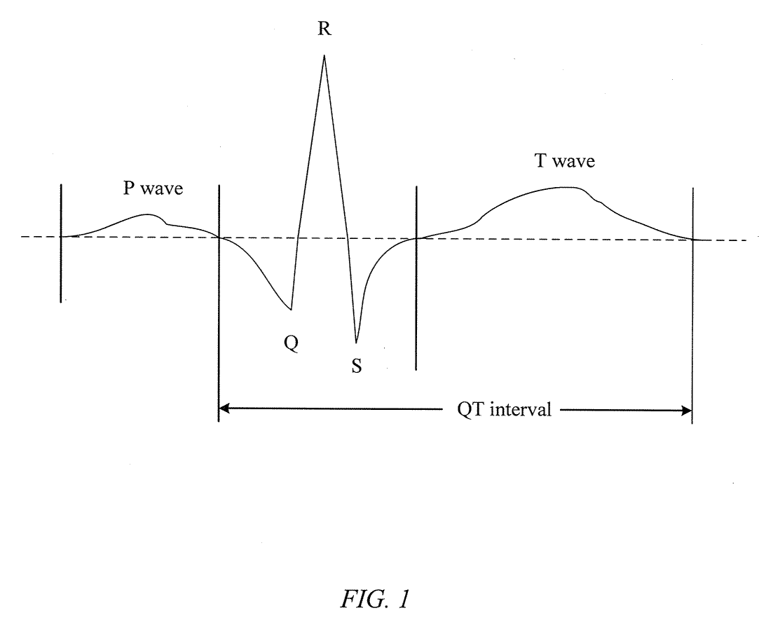

With reference again to FIG. 1, the figure illustrates a typical QT interval in a cardiac cycle detectable by electrodes used in electrocardiography, and in the present invention as well. The QT interval on the ECG is related to the time period from the onset of electrical depolarization (onset of Q wave on ECG) to the end of electrical repolarization (end of T wave on ECG) and is called “electrical systole.”

The QT interval varies according to heart rate as well as other physiologic influences. The QTc interval (QT corrected for heart rate) may be derived by simple mathematical correlation of the QT interval and the heart rate. One method for calculating QTc is application of the Bazett formula. Many other algorithms may also be used, such as the nomogram method, the Frederica formula, and the linear regression equation.

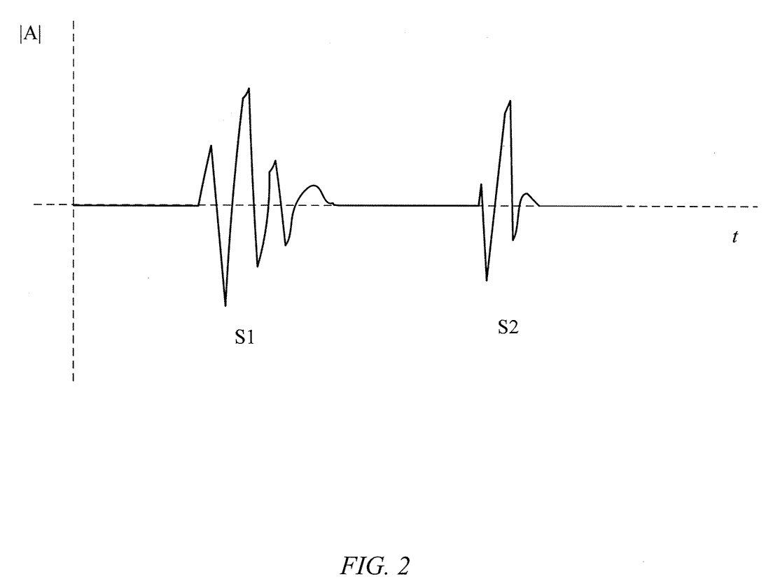

A typical phonocardiograph is illustrated in FIG. 2, which shows the S1 and S2 acoustic pulses in a single cardiac cycle. The horizontal axis represents time, and the vertical axis represents acoustic amplitude (|A|) or a voltage signal picked up by a microphone. The two pulses S1 and S2 represent the familiar 'lub-dub” sound of the human heart as heard through a stethoscope. The QS2 interval is a time interval from the onset of the Q wave on the ECG to the onset of the second heart sound S2 as recorded by a phonocardiogram. The QS2 interval represents the time of mechanical activity of the heart, referred to as “mechanical systole.”

In normal subjects there is a close relationship between the electrical (QT) and the mechanical (QS2) systole. According to research conducted by the inventor of the present disclosure, the QT/QS2 relationship has been determined in more than one thousand resting and exercise ECG tests on LQTS subjects and on normal subjects. The results of these tests provide empirical data from which an algorithm has been developed according to the invention for diagnosing whether a patient has LQTS, whether inherited or acquired, based on the patient's QT and QS2 intervals. Generally, the algorithm involves calculating the QT/QS2 ratio, and comparing the result to the empirical data. The following description includes several exemplary embodiments of systems and methods according to the invention for calculating the QT/QS2 ratio.

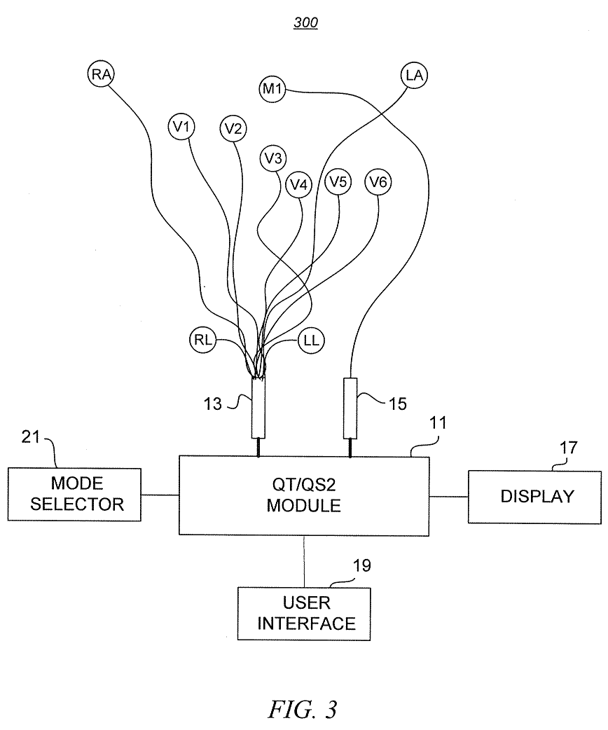

FIG. 3 illustrates one such embodiment of a system 300 for diagnosing LQTS. The system includes a QT/QS2 module 11 configured to receive input signals representing a patient's cardiac activity, and to calculate, based on those signals, the patient's QT/QS2 ratio. The QT/QS2 module 11 may be further configured to acquire the QT and QS2 recordings simultaneously, to ensure that a particular set of QT and QS2 data relates to the same heart beat or to a same cardiac cycle. This will ensure that QT and QS2 measurements are indeed comparable from a physiologic standpoint. In one embodiment, QT/QS2 module 11 may be a personal computer.

In system 300, QT/QS2 module 11 receives input through one or more cable interfaces 13 and 15. Each interface may direct one or more signal-bearing cables to electronics housed within QT/QS2 module 11. A set of cables directed through an interface 13 or 15 may include one or more of electrode cable, microphone cable, ground cable, power cable, or other signal cables. Electrode cables routed through cable interfaces 13 and 15 may include one cable per electrode. In one embodiment, the electrodes may form a set typically used for ECG procedures, such as a 12-lead set having as few as five and as many as ten wires. System 300 is shown having ten electrode wires that form a typical 12-lead set, terminating in a standard set of electrodes that includes four limb electrodes and six chest electrodes. The limb electrodes are: RA (right arm), RL (right leg), LA (left arm), LL (left leg). The chest electrodes are V1, V2, V3, V4, V5 and V6. One or more microphone cables may also be routed through interface 13 or 15, one cable for each microphone connected to the patient. System 300 shows one microphone cable coupled to a microphone M1.

Electrode connections may be made in the same way they are made when operating an ECG machine, for example, by taping each electrode to a designated location. There are many technical standards for 12 lead diagnostic ECG machines, and standard formats for application of the electrodes to the patient by anatomic position. Such standards also specify connection of the wires from the patient electrodes to the ECG machine. Any of these standard procedures may be followed when connecting electrodes RA, RL, LL, LL and V1-V6 without departing from the scope of the present invention.

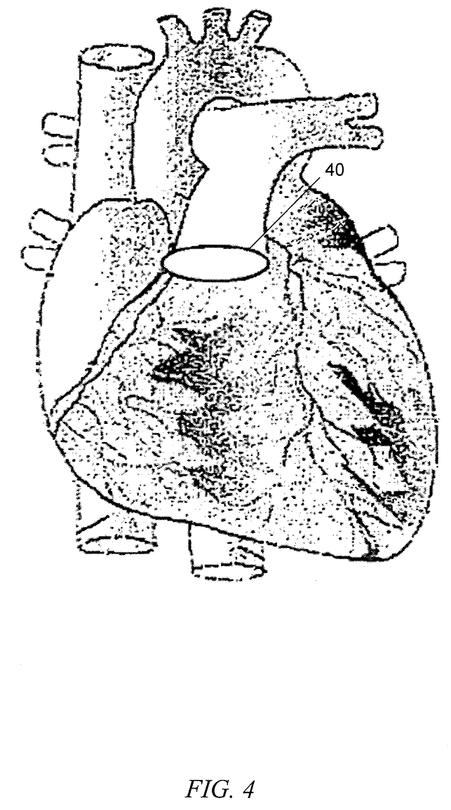

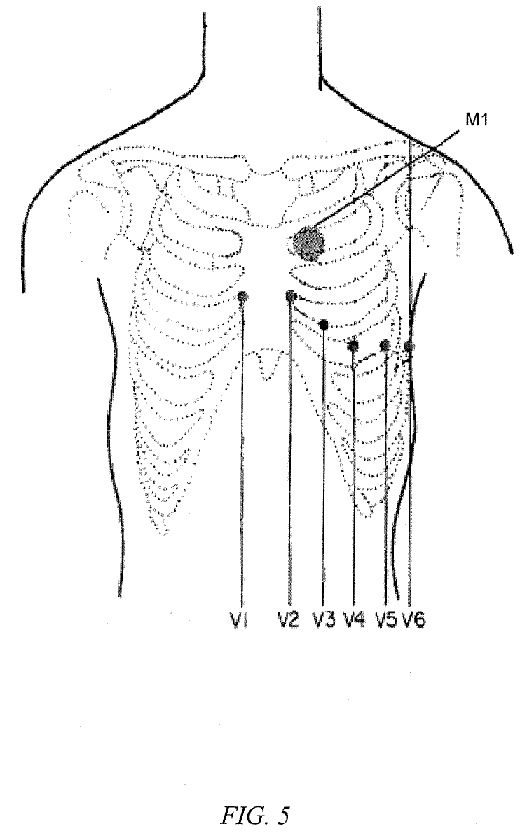

The microphone M1 may be any microphone suitable for use with a phonocardiogram, and may be connected to the patient by any reliable means such as by suction cup or tape to the anterior chest to pick up sound from the aortic and pulmonary valves. FIG. 4 shows the approximate position of the pulmonary valve in a human heart, indicated by the white oval region 40. The sound of interest to be picked up by the microphone is the second heart sound S2 caused by closure of the aortic and pulmonary valves at the end of. Sound from the aortic and pulmonary valves is typically best heard and recorded from near the second left intercostal space, adjacent to the sternum. FIG. 5 shows the location of the second left intercostal space with respect to the anterior chest region of a patient. This location corresponds to a location that is most often optimal for fixing the microphone.

Due to variations in chest anatomy and heart position relative to the chest from one patient to the next, the best S2 signal should be determined by the system operator. To assist the operator, system 300 may include a display unit 17 coupled to QT/QS2 module 11. Display unit 17 may be any or all of a printer, a CRT, an LCD, a plasma screen, or other monitor or output device capable of displaying ECGs and phonocardiograms. In one embodiment, display unit 17 may be a strip chart printer operating between about 25 mm/s and about 500 mm/s. In another embodiment, display unit 17 may output a strip chart at about 100 mm/s. In another embodiment, system 300 may have a selectable recording speed output so that an operator may customize the output of display unit 17 according to personal preference.

By observing the acoustic waveform output from display unit 17, the operator may adjust the position of the microphone and the acoustic filtering characteristics as needed to obtain a suitable S2 size and form for optimal measurements. Display 17 may also include instructions for operating the system, and may display heart rate, QT/QS2 ratios, warning signals, and other data.

A user interface 19 coupled to QT/QS2 module 11 may also be provided. User interface 19 may be a keyboard, mouse, or other input device that allows an operator to operate the module, enter data into the module, or load software into the module. One example of software that may be loaded into QT/QS2 module 11 through user interface 19 is program containing an algorithm for calculating a QT/QS2 ratio.

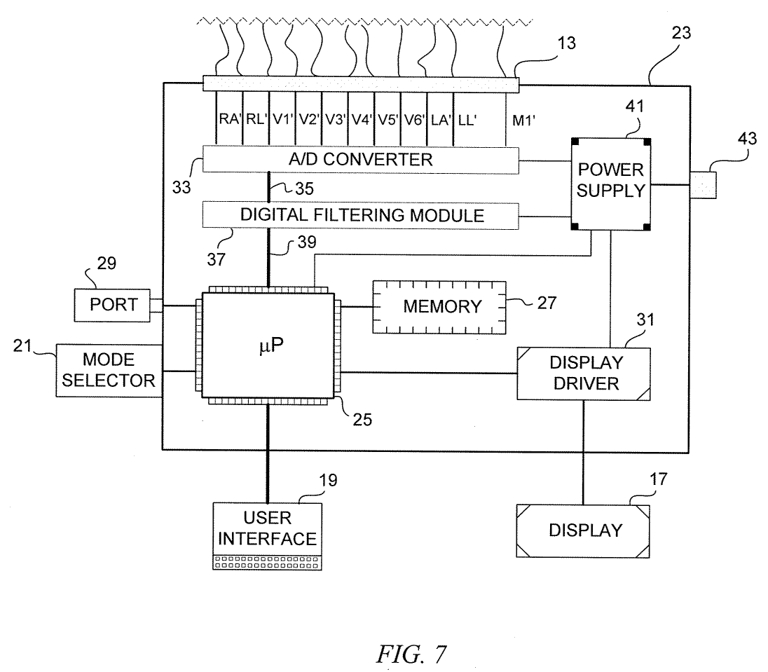

In another embodiment, system 300 may include a mode selector 21 coupled to QT/QS2 module 11. Mode selector 21 may be a switch or other device capable of assuming at least two or three different states, or capable of outputting at least two or three different signals, depending on the number of selectable modes. In a first state, mode selector 21 causes QT/QS2 module 11 to operate as an electrocardiograph. When the first state is selected, QT/QS2 module 11 may be advantageously operated in electrocardiograph mode as a standard electrocardiograph or ambulatory monitor capable of performing the usual 12 lead ECG. In a second state, mode selector 21 causes QT/QS2 module 11 to operate in LQTS diagnostic mode as a device for diagnosing LQTS. In one embodiment, when the second state is selected, QT/QS2 module 11 may simultaneously record ECG leads and a phonocardiogram signal. In another embodiment, when the second state is selected, QT/QS2 module 11 synchronizes the presentation of the electrical and mechanical systoles shown on display unit 17 (see, e.g. FIG. 7). In another state, mode selector 21 causes QT/QS2 module 11 to operate in a phonocardiograph mode as a standard phonocardiograph. Thus, mode selector 21 makes system 300 a more versatile device for a user, in that only one device need be acquired to equip a health care provider with electrocardiograph, a phonocardiograph, and an LQTS diagnostic tool, or any two of the three.

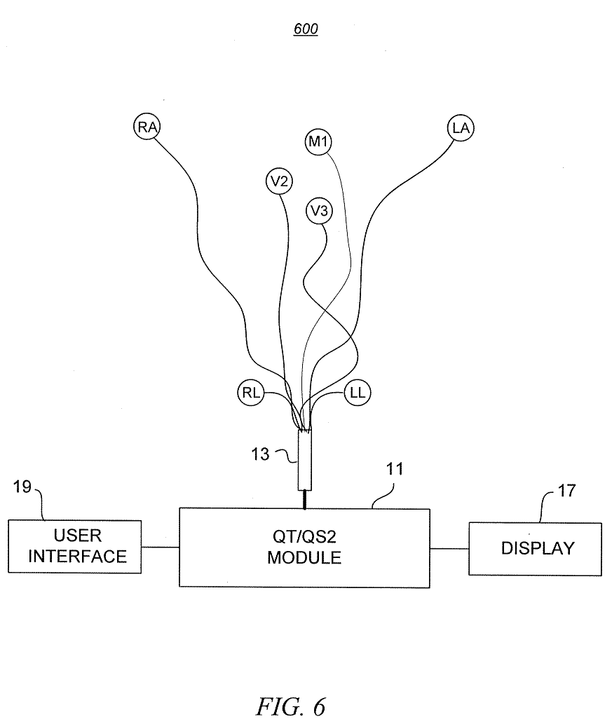

FIG. 6 shows an embodiment of a system 600 according to the invention for diagnosing LQTS. System 600 includes a QT/QS2 module 11 couple to a cable interface 13, a display 17, and a user interface 19. These components operate as in the previous system embodiment, except that user interface 19 may include the mode selector function of mode selector 21.

System 600 uses a fewer number of cables than system 300, in that the ECG electrodes needed for this device are a minimum set required for calculating the QT/QS2 ratio. The minimum set monitors the depolarization and repolarization of the heart ventricles, and provides for derivation of the electrical systole (QT interval) and, along with at least one microphone M1, derivation of the mechanical systole. In one embodiment, the minimum set of electrodes are those needed for recording the standard ECG leads II, V2 and V3.

FIG. 7 shows one embodiment of a QT/QS2 module 23 in greater detail. Central to QT/QS2 module 23 is a microprocessor 25 coupled to a memory or memory devices 27. Microprocessor 25 may be any commercial microprocessor or microcontroller employed in modern computers or digital electronic control systems. Memory device(s) 27 may be any conventional means for storing electronic data, such as ROM, RAM, EEPROM, disk memory, flash memory, or other solid state, magnetic or optical data storage components. Memory 27 may also be integral to the microprocessor 25. Microprocessor 25 controls the operation of the QT/QS2 module 23 by receiving, processing, and outputting signals to and from other electronic components or modules that make up QT/QS2 module 23 or interface therewith.

A user or operator of QT/QS2 module 23 may access the module or enter command input thereto through a user interface 19, a mode selector 21, or an I/O port 29. The I/O port 29 may be any conventional transceiving port such as a serial port, parallel port, network port, RS-232 port, RS-485 port, USB port, or the like. In one embodiment, I/O port 29 may be used to output LQTS diagnostic test results to a printer or to another computer. In another embodiment, I/O port 29 may be used as a path for loading software, such as operating software or executable software, or for loading data, such as empirical LQTS data, into memory 27 for later retrieval or execution by microprocessor 25.

QT/QS2 module 23 may also include a display driver 31 for interfacing with one or more display units 17. Display driver 31 enables output from microprocessor 25 to be displayed using the correct video signal protocol. In one embodiment, the QT/QS2 module 23 may operate in a mode whereby it commands display unit 17 to display a static video screen showing one or more of an ECG, a phonocardiogram, an ECG synchronized with a phonocardiogram, an ECG superimposed on a phonocardiogram, a numerical value representing a QT interval, a numerical value representing a QS2 interval, and a numerical value representing the QT/QS2 ratio. The displayed ECG and/or phonocardiogram preferably correspond to the same cardiac cycle, and one or more cardiac cycles may be statically displayed. In another embodiment, ECG and phonocardiograms may be statically displayed or dynamically displayed on display unit 17, for example, by selecting a desired display mode via user input. Video display graphics may be selectable so that a user may select a desired scale graduation, e.g. 25 mm/s, 50 mm/s, 100 mm/s, or 500 mm/s recording speed.

Signal input to QT/QS2 module 23 is received through one or more cable interfaces 13, from a set of electrodes and at least one microphone. The electrode signals may be received via cables RA′, RL′, V1′, V2′, V3′, V4′, V5′, V6′, LA′ and LL′, each coupled to a corresponding electrode affixed to a patient for measuring the electrical systole and other electric potentials of interest. The microphone signal may be received via a single microphone cable M1′ coupled to the microphone M1 that is used for detecting the second heart sound. Additional microphone inputs may be provided.

The electrode and microphone signals are received by an A/D converter module 33. This module may be any conventional A/D converter, or combination of converters, commonly used in electronic circuit design. The A/D converter module 33 may sample analog signals received from the electrodes and microphone at an appropriate sampling rate to avoid aliasing and to optimize advantages of digital filtration. In one embodiment, the sampling rate may be anywhere between about 10 Hz and about 1000 Hz. In another embodiment, the sampling rate may be selected at about 500 Hz. The A/D converter module 33 may then output the corresponding digital signals (e.g. as bit streams) via bus 35 to a filtering module 37. At filtering module 37, each digital signal may be filtered and/or amplified using appropriate electronics, to reduce noise and normalize the signal levels. In one embodiment, A/D converter module 33 and digital filtering module 37 may be integrated in a single signal conditioning module.

The output signals from digital filtering module 37 are transmitted to microprocessor 25 via bus 39. In one embodiment, each digital signal representing information from an electrode or microphone is received at a separate input of microprocessor 25. In another embodiment, one or more of the digital signals may be multiplexed and received at a common input of microprocessor 25. The signals thus received provide input necessary for calculating the QT interval, the QTc interval (if desired), the QS2 interval, and for producing ECG or phonocardiogram images on display 17. Microprocessor 25 may process the information received by executing algorithms stored in memory 27, and outputting the results to display 17 via display driver 31, or to I/O port 29.

Wireless embodiments are also possible within the scope of the invention. For example, electrode, microphone, or other input signals capable of being transmitted wirelessly may be received at interface 13 wirelessly, according to known techniques. Similarly, output from QT/QS2 module 23 may be transmitted wirelessly to a display 17 or to another receiver. Detailed disclosure of electronic systems required for effecting wireless transmission and reception are well known in the engineering arts, and are not provided herein.

QT/QS2 module 23 may further include a power supply 41. In a portable embodiment of the invention, power supply 41 may be a battery or battery pack, and may include a battery charger connectable to an AC power supply through a power port 43. Alternatively, in a portable or non-portable embodiment, power supply 41 may be an AC-to-DC converter receiving AC power through power port 43. In general, power supply 41 provides DC power necessary for energizing microprocessor 25, display driver 31, digital filtering module 37, A/D converter 33, and any other DC loads within the module.

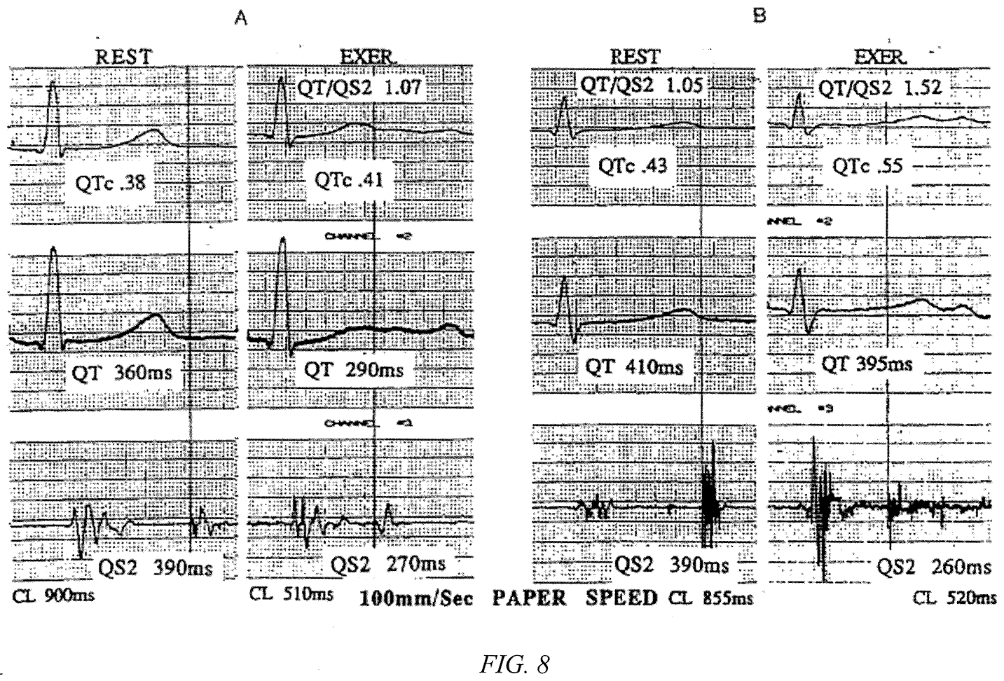

FIG. 8 shows examples of the combined ECG and phonocardiogram images and numerical data recorded by a system according to the invention. Two sets of three graphs are shown on the left-hand side of the figure under the heading A. The graphs under heading A correspond to measurements taken from a patient having a normal QT interval. Two more sets of graphs are shown on the right-hand side of the figure under the heading B. The graphs under heading B correspond to measurements taken from a patient having the LQT1 form of LQTS. Under both heading A and heading B, the top four graphs show ECG recordings, and the two lower graphs are phonocardiogram recordings. Under heading A, the graphs in the left-most column were recorded while the patient was in a state of rest, and the graphs in the right-most column were recorded while the patient was in a state of exercise.

This first signal cluster on each phonocardiogram is an S1 heart sound, and the second signal cluster is an S2 heart sound indicating closure of the aortic and pulmonary valves. Normally, the end of the T wave occurs at near the same time as the beginning of S2. Note that under heading A, from a normal patient, the beginning of S2 is indicated by the vertical line drawn though all three panels. While at rest, the end of the T occurs before the beginning of S2. While at exercise, the end of the T occurs just at or slightly beyond the beginning of S2. These graphs demonstrate the close relationship of QT and QS2 at rest and at exercise in normal patients.

In many patients with abnormal repolarization similar to LQTS, the QT at rest is slightly longer than QS2, thus the QT/QS2 ratio is slightly above 1.0. In other patients the resting QT is longer and thus the QT/QS2 ratio is much larger. In a state of exercise, the QT/QS2 ratio often becomes even further (and abnormally) prolonged, as shown under heading B. At exercise, the QS2 shortens considerably, as indicated by the vertical line that shows the beginning of S2, and closely reflects how the QT interval should respond. However, in LQTS the QT usually does not shorten at all or not to the same degree, which is evident in the top most graphs in that the end of the T wave occurs much later than the beginning of S2, such that the QT/QS2 ratio increases, in this case from 1.05 at rest to 1.52 during exercise. This is a distinctly abnormal response that may be easily detected using a system according to the invention. This disparity of QT response as compared to the patient's expected and appropriate response defines an abnormal QT interval and repolarization physiology, even when the resting QT interval is in the normal range. On the basis of these findings, a comparison of the QS2 interval to the QT interval in states of rest and exercise may provide a highly effective method for diagnosing LQTS and other abnormal heart arrhythmia.

Although the resting QT/QS2 ratio may be helpful in identifying patients with an underlying disorder of QT physiology, it may not always be definitive. However, an algorithm for diagnosing whether a patient is at high risk of having LQTS may be aided based on empirical data. For example, based on data collected by the inventor, a resting QT/QS2 ratio less than 1.0 indicates a low probability of LQTS. A resting QT/QS2 ratio between 1.08 and 1.15 suggests the possibility of LQTS. A resting or exercise/recovery QT/QS2 ratio greater than 1.16 is highly suspicious for LQTS, and the higher the value the more confident the diagnosis of LQTS. Other empirical data are available for analysis by specific genotype and may be included in the diagnostic algorithms.

Values in between these ranges are indeterminate by themselves, but based on the history and other findings in the patient or family member, may influence the ultimate diagnosis. The resting QT/QS2 ratio may be used for screening populations such as young athletes in a pre-participation exam, or patients to whom a QT prolonging drug might be administered, but is a less definitive test than the exercise QT/QS2 ratio. Based on the empirical data, and on many observations of responses of the QT/QS2 ratio during exercise and recovery, it is evident that the exercise QT/QS2 ratio is much more sensitive and specific and is applicable for both individual patient and population screening purposes.

Thus, a method according to one embodiment of the invention for diagnosing LQTS is an analysis of the QT/QS2 ratio at both rest and at exercise. A suitable and preferred exercise protocol, such as could be administered using a treadmill or stationary cycle, may be included in the method. In one embodiment, a patient may be tested first at rest to establish a baseline QT/QS2 ratio, and then tested after undergoing an exercise protocol. A system according to the invention may then calculate a QT/QS2 ratio at various stages during and after exercise. In another embodiment, a patient may be tested only in a state of exercise, and a QT/QS2 ratio calculated.

In evaluating any of the above tests, a threshold value may be established based on empirical data for determining whether a calculated QT/QS2 ratio indicates a high probability of LQTS. For example, the threshold may be set at 1.16. Then, a normal test result may be determined for any values of the QT/QS2 ratio that are less than or equal to 1.16 during exercise and recovery. An abnormal test result may be determined for a QT/QS2 ratio greater than 1.16 on at least two contiguous time period measurements during exercise or recovery. Other similar thresholds and conditions may be established without departing from the scope of the invention.

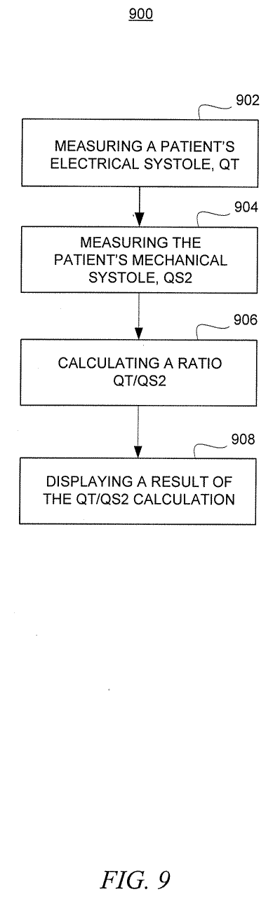

Many embodiments of methods according to the invention are possible. FIG. 9 is one such embodiment 900, showing a process flow diagram for diagnosing LQTS. Method 900 begins at step 902, in which a patient's electrical systole, QT, is measured. For example, the QT interval may be measured using an electrocardiograph, or using a system according to the invention. In the next step 904, the mechanical systole, QS2 is measured. This step may be performed concurrently with step 902. It may be accomplished using one of the foregoing system embodiments, or by using a phonocardiograph or other device for measuring closure of the aortic and pulmonary valves, in combination with detection of the onset of the Q wave, to obtain an accurate measure of the QS2 interval. In the next step 806, the QT/QS2 ratio is calculated. In the final step 908, the result of the calculation for the QT/QS2 ratio is displayed.

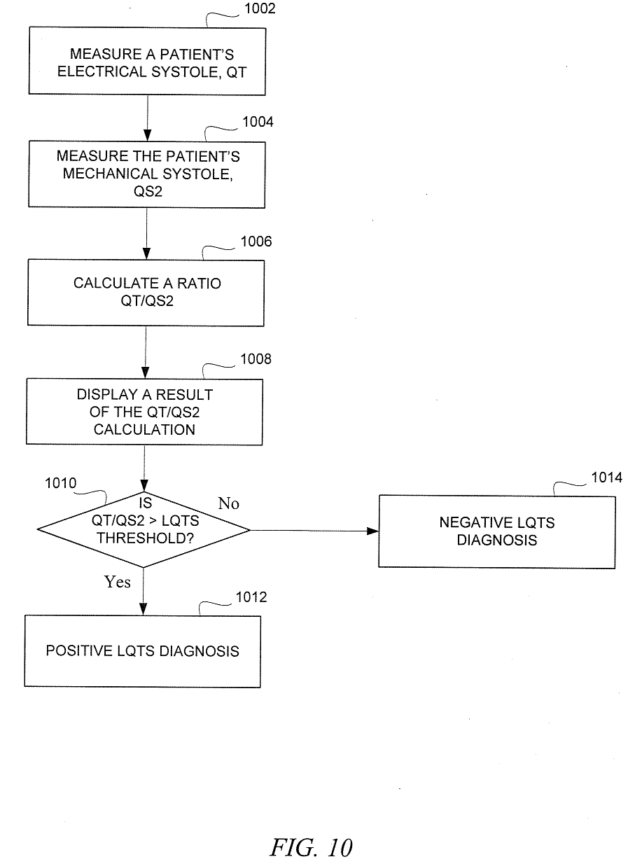

FIG. 10 shows another embodiment of a method 1000 according to the invention for diagnosing LQTS. In the first step 1002, the electrical systole QT is measured. In the next step 1004, the patient's mechanical systole, QS2, is measured. Step 1004 may be performed simultaneously with step 1002. The next step is step 1006, in which the QT/QS2 ratio is calculated. Once that value is obtained, it may be displayed in the next step 1008, for example, on a computer monitor or printout. Then, in decision block 1010 the QT/QS2 ratio is compared to an LQTS threshold value. In one embodiment, the LQTS threshold may be established based on empirical data. In the decision block 1010 a determination is made whether the QT/QS2 ratio exceeds the LQTS threshold. If so, the process ends at final step 1012, which represents a positive LQTS diagnosis, or alternatively, a high probability indication of LQTS. If not, the process ends at final step 1014, which represents a negative LQTS diagnosis, or low probability indication of LQTS. In either final step, the indication may be made on a display unit or computer monitor.

In another method according to the invention, patients may be screened prior to administering a known QT prolonging drug. Baseline cardiac repolarization properties may be assessed using the invention to identify those patients that may have an abnormal underlying repolarization substrate and, thus, an increased risk for an arrhythmia when given a prescription drug known to potentially cause heart arrhythmia. For example, if a calculated QT/QS2 ratio is high (e.g. at 1.08 or above), the patient may be disqualified as a candidate for receiving the drug. Determination of the degree or magnitude of QT interval prolongation after the administration of the drug allows for more reliable risk assessment for cardiac arrhythmias while on the drug. The examination (e.g. method 900) may be performed at each incremental dose of the QT prolonging medication to determine any further degree of QT prolongation that would affect risk for arrhythmia.

In another embodiment of a method according to the invention, an initial step may be added to a method such as method 900 or method 1000 wherein a pharmaceutical drug or other bioactive agent may be administered to a test subject. At predetermined time periods, a QT/QS2 ratio may be determined in order to assess whether the drug or agent may cause side effects of QT interval prolongation. Such a method may be especially useful in drug efficacy tests, such as those conducted by independent test laboratories or the FDA.

In another embodiment of a method according to the invention, members of an at-risk population, such as young persons seeking to pursue athletics, may be screened for LQTS or other life-threatening cardiac arrhythmias. The screening may also be implemented as part of a pre-participation exam for athletes, policemen, firemen, emergency response personnel, and others who may experience rapid and significant exercise or emotional stress.

The invention may also be employed to evaluate patients who have experienced loss of consciousness to discriminate between relatively benign causes (such as vasovagal syncope or neurocardiogenic syncope and related conditions such as postural or hypotensive orthostatic tachycardia syndrome) and those that are due to dangerous cardiac arrhythmias such as inherited and acquired LQTS. It may also be used for diagnostic evaluation of patients suspected by symptoms or family history to have inherited LQTS.

In another method according to the invention, steps of method 900 or 1000 may be used to optimize treatment of LQTS patients. According to data acquired by the inventor, treatment efficacy may be demonstrated by reduction in the QT/QS2 ratio, with reduction to a normal value being an optimal response. In one example, a patient may be periodically evaluated by measuring a QT/QS2 ratio. If the ratio changes over time, appropriate action may be taken such as prescribing a new medication or changing dosage levels. In another example, the method may be used before and after trial dosages of medications to assess whether the patient experiences a favorable or unfavorable reaction to the medication in terms of his QT/QS2 interval.

A method according to the invention may also be implemented to investigate the additive predictive value for risk of sudden death in patients with known structural heart disease, such as cardiomyopathy, valvular and coronary heart disease, and presumed increased risk of death. The invention may also aid in the identification of different genetic subtypes of inherited LQTS, and differentiation of inherited LQTS from other causes of QT interval prolongation such as structural heart disease, and other investigational studies of repolarization properties in LQTS and related arrhythmogenic diseases.

The invention has been disclosed in an illustrative style. Accordingly, the terminology employed throughout should be read in an exemplary rather than a limiting manner. Although minor modifications of the present invention will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

What is claimed is:

1. A system for diagnosing LQTS in a patient, comprising:
electrodes for detecting an electrical systole, QT, in the patient;
at least one microphone for detecting a mechanical systole, QS2, in the patient;
a processing module receiving the detected electrical systole and the detected mechanical systole, calculating a QT/QS2 ratio, and outputting the ratio as a diagnostic result.

2. The system of claim 1 further comprising a display unit coupled to the processing module for displaying the QT/QS2 ratio.

3. The system of claim 2 wherein the display unit displays a first waveform representing the electrical systole and a second waveform representing the mechanical systole.

4. The system of claim 1 further comprising a mode selector for selecting between first and second operating modes of the processing module, the processing module in the first operating mode operating as an electrocardiograph, the processing module in the second operating mode calculating the QT/QS2 ratio.

5. The system of claim 1 wherein the electrodes comprise a minimum of ECG leads II, V2 and V3.

6. The system of claim 1 further comprising a user interface coupled to the processing module.

7. The system of claim 1 wherein the processing module further comprises an analog-to-digital converter receiving analog input from the electrodes and the at least one microphone.

8. The system of claim 7 wherein the processing module further comprises a signal conditioning module receiving digital output from the analog-to-digital converter.

9. The system of claim 8 wherein the processing module further comprises a microprocessor coupled to memory.

10. The system of claim 9 wherein the microprocessor calculates the QT/QS2 ratio based on output from the signal conditioning module.

11. The system of claim 10 wherein the microprocessor calculates the QT/QS2 ratio by executing an algorithm stored in the memory.

12. The system of claim 10 wherein the microprocessor compares the QT/QS2 ratio to a threshold value stored in the memory.

13. The system of claim 12 wherein if the QT/QS2 ratio exceeds the threshold value, the microprocessor outputs an alarm.

14. The system of claim 13 further comprising a display unit, wherein the microprocessor outputs the alarm to the display unit.

15. The system of claim 1 wherein the detected electrical systole and the detected mechanical systole received by the processor for calculating the QT/QS2 ratio occur during a same cardiac cycle.

16. The system of claim 1 wherein the electrical systole comprises a time interval during one period of a cardiac cycle from onset of electrical depolarization of heart ventricles to end of electrical repolarization of the heart ventricles.

17. The system of claim 1 wherein the mechanical systole comprises a time interval during one period of a cardiac cycle from onset of electrical depolarization of heart ventricles to occurrence of second heart sound S2.

18. A module for diagnosing LQTS in a patient, comprising:
electrode inputs for receiving electrode signals;
at least one microphone input for receiving an audio signal;
a microprocessor receiving the electrode inputs and the at least one microphone input, deriving therefrom an electrical systole QT and a mechanical systole QS2, calculating a QT/QS2 ratio; and outputting the ratio as a diagnostic result.

19. The module of claim 18 further comprising a memory coupled to the microprocessor.

20. The module of claim 19 wherein the microprocessor derives the electrical and mechanical systoles by executing an algorithm stored in the memory.

21. The module of claim 19 wherein the microprocessor calculates the QT/QS2 ratio by executing an algorithm stored in the memory.

22. The module of claim 18 wherein the microprocessor calculates the QT/QS2 ratio from electrical and mechanical systoles derived from a common cardiac cycle.

23. A method for diagnosing LQTS in a patient, comprising:
measuring an electrical systole, QT, in the patient;
measuring a mechanical systole, QS2, in the patient;
calculating a QT/QS2 ratio; and
displaying a result of the QT/QS2 calculation.

24. The method of claim 23 wherein the electrical systole is measured using one or more electrodes.

25. The method of claim 23 wherein the mechanical systole is measured using one or more microphones.

26. The method of claim 23 further comprising simultaneously displaying a first waveform representing the electrical systole and a second waveform representing the mechanical systole.

27. The method of claim 26 further comprising synchronizing the first and second waveforms.

28. The method of claim 23 wherein the result comprises a number.

29. The method of claim 23 further comprising comparing the result to a predetermined threshold.

30. The method of claim 29 further comprising displaying a diagnosis based on the comparison.

31. The method of claim 23 further comprising measuring the electrical and mechanical systoles while the patient is in a state of rest.

32. The method of claim 31 further comprising repeating all steps while the patient is in a state of exercise.

33. The method of claim 23 further comprising measuring the electrical and mechanical systoles while the patient is in a state of exercise.

34. The method of claim 23 wherein the electrical systole comprises a time interval during one period of a cardiac cycle from onset of electrical depolarization of heart ventricles to end of electrical repolarization of the heart ventricles.

35. The method of claim 23 wherein the mechanical systole comprises a time interval during one period of a cardiac cycle from onset of electrical depolarization of heart ventricles to closure of a pulmonary valve.

36. The method of claim 23 wherein the measuring steps occur during a same cardiac cycle.

37. A method for evaluating the effectiveness of a drug for treating LQTS in a patient, comprising:
(a) measuring an electrical systole, QT, in the patient;
(b) measuring a mechanical systole, QS2, in the patient;
(c) calculating a QT/QS2 ratio;
(d) recording a result of the calculated QT/QS2 ratio;
administering a desired dose of the drug to the patient; and
repeating steps (a) through (d).

38. The method of claim 37 wherein QT and QS2 are measured during a same cardiac cycle.

39. A method for optimizing a dosage of a drug for treating LQTS in a patient, comprising:
(a) measuring an electrical systole, QT, in the patient;
(b) measuring a mechanical systole, QS2, in the patient;
(c) calculating a QT/QS2 ratio;
(d) recording a result of the calculated QT/QS2 ratio;
administering a dose of the drug to the patient;
repeating steps (a) through (d); and
comparing the recorded results to determine effectiveness of the dose.

40. The method of claim 39 wherein QT and QS2 are measured during a same cardiac cycle.

41. A method for evaluating whether a drug poses a risk of causing acquired LQTS in a patient, comprising:
(a) measuring an electrical systole, QT, in the patient;
(b) measuring a mechanical systole, QS2, in the patient;
(c) calculating a QT/QS2 ratio;
(d) recording a result of the calculated QT/QS2 ratio;
administering a dose of the drug to the patient;
repeating steps (a) through (d); and
comparing the recorded results.

42. The method of claim 41 wherein QT and QS2 are measured during a same cardiac cycle.
